(19) United States
(12) Patent Application Publication
(10) Pub. No.: US 2011/0091879 A1
(43) Pub. Date: Apr. 21, 2011

(54) MOBILE DEVICE FOR ISOLATION OF NUCLEIC ACIDS

(75) Inventors: Timo HILLEBRAND, Hoppegarten (DE); Claus KNIPPSCHILD, Jena (DE); Benjamin JASCHINSKY, Halle (DE)

(73) Assignee: AJ INNUSCREEN GmbH, Berlin (DE)

(21) Appl. No.: 12/857,299

(22) Filed: Aug. 16, 2010

Related U.S. Application Data
(63) Continuation of application No. PCT/EP2009/051820, filed on Feb. 16, 2009.

(30) Foreign Application Priority Data
Feb. 15, 2008 (DE) 10 2008 009 920.1

(51) Int. Cl. C12Q 1/68; C12M 1/33; C12M 1/34

(52) U.S. Cl. 435/6; 435/306.1; 435/287.2

(57) ABSTRACT

The invention relates to a mobile device system, comprising a hand-held device and a test kit for the mobile isolation of nucleic acids. The hand-held device comprises at least one sample block for inserting sample containers, a sample block holder with boreholes or recesses for accommodating the sample blocks, a device base with electronic control units for the sample blocks, a voltage source and a connection to the sample block holder, as well as a test kit for the isolation of nucleic acids. The hand-held device is characterized in that the sample blocks can be removed from the sample block holder and the sample block holder can be removed from the device base.

### CROSS-REFERENCE TO RELATED APPLICATIONS

This application claims benefit under 35 U.S.C. §365(c) and 35 U.S.C. §120 to PCT/EP/2009/051820, filed Feb. 16, 2009. Priority is also claimed to DE 10 208 009 920.1, filed Feb. 15, 2008. Both of these applications are incorporated by reference in their entireties.

### BACKGROUND OF THE INVENTION

1. Field of the Invention

The subject matter of the invention is a mobile device system comprising a hand-held unit and a test kit for mobile isolation of nucleic acids.

2. Description of the Related Art

The investigation of diagnostically relevant biological samples such as serum, plasma, blood, swab samples or organ triturations for detection of infectious pathogens has taken on enormous importance in recent years. Virus infections such as HIV, HCV or HBV are spreading worldwide. Furthermore, bacterial infections are again spreading, as the result of beginning climatic changes among other reasons. The advent of new, deadly infectious diseases with extremely high infection potential (SARS, bird flu) is showing ever more clearly that a rapid diagnosis that can be performed on the spot is decisive for the prevention of epidemics. This also concerns the problem of bioterrorism and rapid on-the-spot detection of pathogens that may be used as biological weapons.

Several developments in what is known as on-the-spot diagnosis exist for dealing with this problem. Supposedly they relate to devices that combine all steps of molecular diagnosis (isolation of nucleic acids, amplification and detection). These developments are focused on the area of military diagnostics and, by analogy with the traditional “REAL TIME PCR methods”, are very expensive, both in terms of equipment and of reagents.

It appears realistic that the steps of amplification and detection can be solved. However, the greatest problem lies in integrating the preparation of samples. The reason for this is very simple and is based on the fact that the starting materials to be investigated are extremely diverse and in particular are “problematic”. The problem of on-the-spot isolation of nucleic acids is not solved in this way.

Under traditional conditions, DNA is isolated from cells and tissues by digesting the nucleic acid-containing starting materials under strongly denaturing and reducing conditions, in some cases also with the use of protein-decomposing enzymes, then purifying the resulting nucleic acid fractions via phenol/chloroform extraction steps and separating the nucleic acids from the aqueous phase by means of dialysis or precipitation by ethanol (Sambrook, J., Fritsch, E. F. and Maniatis, T., 1989, CSH “Molecular Cloning”). These “traditional procedures” for isolating nucleic acids from cells and especially from tissues are very time-consuming (sometimes longer than 48 hours), and they require a considerable outlay for apparatus. Various alternative procedures for isolating nucleic acids from different biological starting materials make it possible to circumvent the complex and health-endangering phenol/chloroform extraction of nucleic acids and to shorten the time required. All of these procedures are based on a method developed and described for the first time by Vogelstein and Gillespie (Proc. Natl. Acad. Sci. USA, 1979, 76, 615-619) for preparative and analytical purification of DNA fragments from agarose gels. The method combines dissolution of the agarose containing the DNA bands to be isolated in a saturated solution of a chaotropic salt (NaI) with binding of the DNA to glass particles. The DNA fixed on the glass particles is then washed with a detergent solution (20 mM Tris HCl [pH 7.2]; 200 mM NaCl; 2 mM EDTA; 50% v/v ethanol) and then detached from the carrier particles.

To date, this method has undergone several modifications, and at the present time it is being used for different procedures of extraction and purification of nucleic acids from different sources (Marko, M. A., Chipperfield, R. and Birnboim, H. G., 1982, Anal. Biochem., 121, 382-387).

Furthermore, numerous reagent systems are now available worldwide, especially for purification of DNA fragments from agarose gels and for isolation of plasmid DNA from bacterial lysates and even for isolation of longer-chain nucleic acids (genomic DNA, cellular total RNA) from blood, tissues or else cell cultures. All of these commercially available kits are based on the sufficiently well-known principle of binding of nucleic acids on mineral carriers in the presence of solutions of different chaotropic salts, and they use suspensions of finely ground glass powder (such as Glasmilk, BIO 101, La Jolla, Calif.), diatomaceous earths (Sigma Co.) or else silica gels (Diagen, DE 4139664 A1).

A procedure for isolation of nucleic acids that is practical for numerous different applications is disclosed in U.S. Pat. No. 5,234,809 (Boom). This patent describes a procedure for isolation of nucleic acids from nucleic acid-containing starting materials by incubation of the starting material with a chaotropic buffer and a DNA-binding solid phase. The chaotropic buffers bring about both lysis of the starting material and binding of the nucleic acids on the solid phase. The procedure is very suitable for isolating nucleic acids from small amounts of sample, and finds its practical application especially in the area of isolation of viral nucleic acids.

Specific modifications of these procedures relate to the use of novel carrier materials, which exhibit advantages in application for certain problems (WO 95/34569 A). In more recent patents, it is disclosed that so-called antichaotropic salts can also be used very efficiently and successfully as components of lysis/binding buffer systems (EP 1135479) for the adsorption of nucleic acids on silicate materials known to and used by the person skilled in the art. The advantage of this procedure is that the danger to health posed by the extraction system is greatly reduced by circumventing the use of chaotropic salts. Nevertheless, with a view to achieving the highest possible yield of nucleic acid recovery in the lysis buffer, high salt concentrations (>1.5 M) are still required for efficient isolation of nucleic acids from a complex biological sample. For example, the patent discloses that the lysis buffers suitable for use have salt concentrations between 1.5 M and 3 M.

These few examples from the background art make it clear that a large number of simple procedures is available for isolation of nucleic acids. In particular, the possibility of isolating nucleic acids by adsorbing them on suitable carrier materials (glass-fiber materials in the form of filters, suspensions of silicate components or different kinds of magnetic or paramagnetic particles) has been adopted worldwide in laboratory routine. However, the person skilled in the art is also aware that the performance of isolation of nucleic acids in a manner that in principle is independent of the nature of the chemistry used is always contingent on a well-equipped laboratory. Centrifuges, vortexers, thermomixers or incubators, separation modules for magnetic particles and possibly also automatic systems are among the equipment needed. In order to permit isolation of nucleic acids under field conditions also, and therefore using mobile systems, the only solutions attempted heretofore are those that link the process of isolation of nucleic acids with subsequent amplification and detection. Such an approach is extremely complicated, and also has not yet been solved in a universal form. Furthermore, such equipment systems have a very high purchase price, as do the needed consumable materials. However, simple and above all inexpensive as well as universally usable procedural solutions (relative to the most diverse biological samples) do not yet exist.

### BRIEF SUMMARY OF THE INVENTION

An object of the invention is to eliminate the described disadvantages of the solutions associated with the prior art. This object as well as others have been achieved according to the features described herein. The mobile system (hand-held unit) disclosed herein makes it possible in simple and inexpensive manner to isolate nucleic acids from the most diverse starting materials, even under “field conditions”, and above and beyond this it can also be operated by “non-specialists”.

The combination of mobile miniature device and extraction reagents opens up the possibility, for the first time, that diagnosis of infectious diseases in developing countries can be achieved without qualitative restrictions. The invention solves the described problems in the most ideal way and, in terms of device design and of the resulting simplicity of isolation of nucleic acids from biological samples, is also suitable for use by “non-specialists” under field conditions. Furthermore, not only the equipment but also the needed reagents are extremely inexpensive. This represents an enormous advantage compared with the conceived equipment systems, especially for military applications. The invention solves the existing problem with regard to mobile nucleic acid isolation that can be performed on the spot, and does so surprisingly in very simple form. Aspects of the invention include the following:

A hand-held unit for mobile isolation of at least one nucleic acid, comprising at least one sample block for insertion of sample vessels, a sample block holder with bores or recesses for receiving the sample blocks, a device base with electronic control units for the sample blocks, a voltage supply, a connection to the sample block holder, and, a kit or reagent(s) for the extraction and/or isolation of nucleic acids; wherein the sample blocks can be removed from the sample block holder, and wherein the sample block holder can be removed from the device base. The hand-held unit may be configured so that the sample blocks contain at least one heating module and/or one magnetic separation module and it may contain a magnetic separation module that is equipped with one or more magnets. The hand-held unit may contain one or more viewing windows disposed in the sample block. The sample block holder can contain a shaking or vibrating device or one or more reversible connection points for the exchangeable blocks, or both. In one embodiment the sample block holder contains an additional heating device. The sample blocks in the hand-held device can be connected to the sample block holder and the sample block holder is connected to the device base via plug connectors or a magnetic connection. The hand-held unit preferably does not rely on an external power supply, but can contain a voltage supply that is a self-contained battery, which may be replaceable or rechargable, or accumulator. However, in some embodiments the hand-held device may contain a port for an external voltage supply or a port for recharging a voltage supply contained in the hand-held unit. A hand-held unit can be configured for portability and for ease of use. For example, its dimensions may range between a height between 6 and 10 cm, a depth between 2 and 4 cm, and a width between 4 and 8 cm. Other exemplary, but non-limiting dimensions include a height between 7.5 to 8.5 cm, preferably 8 cm, depth between 2.5 to 3.5 cm, preferably 3 cm, and width between 4.5 to 5.5 cm, preferably 5 cm.

The hand-held unit can contain a heating module that simultaneously serves as a magnetic separation module; wherein a) a recess for receiving a magnet is disposed next to this heating and magnetic separation module, or b) the heating and magnetic separation module contains an energizable electromagnet.

The hand-held unit as described above may contain at least one of the following elements: a) one or more Pasteur pipettes or other disposable pipettes, b) one or more bottles for reagent waste, c) one or more suspensions containing magnetic or paramagnetic particles, d) one or more reaction vessels, e) one or more samples of lysis buffer as well as one or more optional proteolytic enzymes in liquid form or one or more reaction vessels for lysis of the sample containing storage-stable solid formulations of lysis reagents and proteolytic enzymes, f) one or more binding buffers, g) one or more detergent buffers, and h) one or more elution buffer. For example, the unit may comprise components a), b), c) and d) or it may comprise components a), b), c), d), e), f), g) and h). The hand-held unit can comprising e) a lysis buffer and a proteolytic enzyme in liquid form or e) a lysis buffer and a reaction vessel for lysis of the sample containing storage-stable solid formulations of lysis reagents and proteolytic enzymes. The hand-held unit may further comprise at least one means for detecting a target nucleic acid in a nucleic acid isolated by said unit, and means for recording or communicating a result of said detection.

The hand-held unit may be contained within a suitable carrying container, for example, one that is durable and protective under field conditions, but which is easily accessed and cleaned or maintained, which contains the hand-held unit and, optionally, at least one portable diagnostic kit or instrument. The hand-held device itself, a kit containing it, or carrying container for the hand-held unit may contain instructions about how to use it to isolate a target nucleic acid. Means for illumination the device during use may be integrated into the hand-held unit or a kit or carrying container for the unit, such as LEDs or other light elements powered by the voltage-supply. Storage spaces for sample blocks, or containers (e.g., glass or plastic tubes), or substrates (e.g., paper, glass or plastic surfaces) for holding, binding, or absorbing a process target nucleic acid sample, or for arranging or identifying processed target nucleic acid samples may also be included.

In another aspect, the invention involves a method for mobile isolation of nucleic acids by means of the hand-held unit described herein, comprising the following steps: lysing a sample containing a nucleic acid, binding DNA or other nucleic acid in the lysed sample to magnetic particles; separating the magnetic particles containing the bound DNA or other nucleic acid; washing the magnetic particles containing the bound nucleic acid; drying the magnetic particles; eluting the DNA or other nucleic acid from the magnetic particles; thus recovering an isolated nucleic acid sample; and optionally detecting a target nucleic acid sequence in said isolated nucleic acid sample.

### BRIEF DESCRIPTION OF THE DRAWINGS

FIG. 1 shows the inventive device in a preferred embodiment. The heating block and the magnetic separation block can be removed from the sample block holder. The sample block holder contains a shaking device and if necessary heating elements and can be removed from the base device.
List of reference numerals for FIG. 1:
(1) Heating block
(2) Magnetic separation block
(3) heating device
(4) Magnet
(5) Shaking device, if necessary with heating element
(6) Sample block holder
(7) Device base

FIG. 2 shows a schematic diagram of a portable hand-held unit for isolation of nucleic acids in the case of mobile on-the-spot isolation of nucleic acids and of a test kit for performing the extraction procedure. In FIG. 2 the reference numbers define the following elements or components:
(1) Heating module with heating element
(2) Printed circuit board
(3) Accumulator/battery
(4) Vibration plate
(5) Magnetic strip
(6) Separation module
(7) Recess for reaction vessel.

FIG. 3 shows another embodiment of the unit which can share elements (2), (3), (4), (6) and (7) with the embodiment of FIG. 2, but in which reference number (1) describes a separating and heating module having a heating element and reference numeral (5) depicts an energizable electromagnet.

FIG. 4 shows another embodiment of the unit which can share elements (2), (3), (4), (6) and (7) with the embodiment of FIG. 2, but in which reference number (1) describes a separating and heating module having a heating element and reference numeral (5) depicts an insertable magnet.

### DETAILED DESCRIPTION OF THE INVENTION

The invention employs the sufficiently well-known principle of isolation of nucleic acids by means of magnetic or paramagnetic particles. The general sequence of the procedure corresponds to that already described in the prior art. It is subdivided into the following steps:
1. Lysis of the starting material
2. Binding the liberated nucleic acids to magnetic or paramagnetic particles
3. Washing the surface-bound nucleic acids
4. Drying the particles
5. Elution of the bound nucleic acid.

As already described, equipment such as a thermomixer or water bath (for lysis of the sample), a vortexer for thorough mixing of the reagent preparations and a magnetic separator are needed to perform these steps of the procedure under laboratory conditions. Heretofore the described diversity of the starting materials (sample structure and surrounding matrix) has made it impossible to integrate a nucleic acid purification procedure with an analysis procedure.

Although numerous disposable sample vessels exist for treatment of individual matrixes or sample materials, heretofore no mobile system that permits partly automated processing of these various vessels has been available. As an example, such sample vessels are capable of receiving swabs, are equipped with filter membranes or by virtue of their internal shape permit better separation of various sample phases.

The invention described here achieves the stated objects by providing a universal device base, a sample block holder with adapted, functionalized sample blocks (for inserting the sample vessels and consumables), and adapted, easy-to-do purification chemistry. This therefore gives the user the opportunity to achieve a matrix and sample-specific purification of nucleic acids by manual combination of these components and various consumables.

Surprisingly, it is precisely this exchangeable block system that makes it possible to process the entire spectrum of sample material and applications in the field.

The device base contains electronic control units for the blocks, the voltage supply and a connection to the sample block holder.

The sample block holder contains a shaking or vibrating device as well as one or more reversible connection points for the exchangeable blocks. In a further alternative embodiment, this element contains an additional heating device if necessary.

In order to guarantee easy separation of the blocks from the sample block holder or of the sample block holder from the device base, it is conceivable to bring the two elements into contact not with a plug connector but instead by means of a magnetic connection. Under these conditions, the flexibility of the overall device can be considerably increased by the easy removal of the block and of the sample block holder.

One embodiment of the invention includes a sample block with heating module, wherein the geometry of the block is specially adapted to the already described consumable materials. In a further alternative embodiment, the heatable sample block is equipped with a shaking device. The heatable sample block may comprise a suitable heating element, including a voltage-source or battery-operated Peltier element having a heating rate of <5° C./s, 5° C./s, 10° C./s, 12.5° C./s and up to 15° C./s.

Since the greater part of the sample holder container (consumables) is made from a transparent polymer, it is possible for the user, in a further alternative embodiment, to evaluate the purification process visually through a viewing window in the sample block and thus to exert a direct, manual influence on the process. Liquids can be added or removed by means of the disposable pipettes provided in the device system. In this way controlled, safe handling of even highly viscous liquids is guaranteed.

A further block of this exchangeable block system may be a magnetic separation block. This magnetic separation block is characterized in that it can be removed from the sample block holder during the purification process. It is equipped with one or more strong magnets, which are used for separation of the magnetic particles, which migrate to the wall of the consumable. The adhering magnetic particles must now be freed from the liquid. Since the separation block can be detached, easy separation of the liquid can be achieved. For this purpose the user can simply remove the block, turn it over and allow the liquid to drain. The strong magnets immobilize the important magnetic particles, and so no loss is suffered.

In this embodiment also the separation block is provided with viewing windows, so that the user (if he does not wish to turn the vessel upside down) can see into the vessel and also remove a sample of the liquid with a disposable pipette. Thus the user is able to hold the separation block containing a reversibly fixed sample vessel in one hand and to achieve precise removal of the liquid.

Surprisingly, it has been found, for various magnetic particles and various sample matrixes, that the arrangement of the magnets and the strength of the magnets have a very pronounced effect on the separation. It has also been surprisingly found, in the use of an identical consumable and the same arrangement of a given magnet type, that the yield of nucleic acids can differ considerably even for only minor differences in the liquid, such as different viscosities.

Consequently, a considerable improvement of the yields can be achieved by an optimum arrangement of the magnets, even though the strength of the magnet or magnets may not necessarily have a very large influence.

Likewise the user very simply has the possibility of exchanging a separation module and inserting it in the sample block, and so the feature of a heated and shaken separation module if necessary is not lost.

Because of the exchangeability of the separation module and of the knowledge obtained about the arrangement and strength of the magnet or magnets, the most diverse separation modules are conceivable for the most diverse procedures and applications. Likewise adaptation to the most diverse consumables is conceivable while retaining the mobile character of the device.

As an example, a viscous medium containing magnetic particles can be considered here, wherein the arrangement of the magnets should be chosen such that penetration of the lines of force in a manner adapted to the consumable and to the liquid level permits rapid separation to be achieved within a space optimized for this procedure and this matrix.

By the fact that the sample block holder contains a variable shaking device, the functionality of the individual exchangeable blocks can be enhanced. For example, shaking assists separation of the magnetic particles in highly viscous matrixes.

Analysis of the purified nucleic acids prepared by the invention may be achieved by suitable procedures on the spot or in a laboratory. In a further alternative embodiment, the connector between the device base and sample block holder can be configured such that, after the sample block holder has been removed, a suitable analysis device can be mounted and provided with the purified sample. In this way the device base serves as a mobile basic supply device for a further tool system.

The schematic construction of the hand-held unit is schematically illustrated under the exemplary embodiment. In this way the mobile hand-held unit technically combines all of the needed technical specifications, which are satisfied in the laboratory by separate individual devices (thermomixer or incubator, vortexer, magnetic separator). Thus on-the-spot isolation of nucleic acids from different sources can be achieved simply and rapidly with the inventive hand-held unit. The hand-held unit is further combined with a test kit, which contains the reagents needed for standard isolation of nucleic acids as well as disposable plastic material.

The sequence of isolation of nucleic acids by means of the inventive hand-held unit, as implemented for a throat smear, for example, is then as follows under field conditions:

1. Lysis of the Sample:

For this purpose transfer swab into a reaction vessel and add lysis buffer and if necessary a proteolytic enzyme by means of a disposable pipette or by means of a dropper bottle. Optionally the lysis reagent and the enzyme may already be present as storage-stable components in the reaction vessel, so that then only water has to be added to the swab. Transfer the vessel into the hand-held unit (into the recess of the heating block or of the heating/shaking block). Heat the heating block to 30-95° C. (depending on the application) and if necessary shaking the vessel during lysis. For example, the block may be heated to 30, 31, 32, 35, 37, 50, 42, 45, 47, 50, 55, 60, 62, 65, 68, 70, 75, 80, 85, 90, 92, 95, or 95° C. or any intermediate value.

2. Binding of the DNA to Magnetic Particles:

For this purpose add a binding buffer containing magnetic particles to the lysis preparation and shake briefly until binding buffer, magnetic particles and lysis preparation have become completely and thoroughly intermixed.

3. Separation of the Magnetic Particles Containing the Bound Nucleic Acid:

For this purpose insert vessel into the recess of the magnetic separation module. Herein accumulation of the magnetic particles takes place. Remove the remaining liquid by means of a disposable pipette or by extracting the magnetic block and simply decanting into a waste bottle for disposal.

4. Washing the Magnetic Particles Containing The Bound Nucleic Acid:

For this purpose use a disposable pipette or dropper bottle to add a detergent buffer to the reaction vessel containing the separated magnetic particles. Shake the reaction vessel briefly and separate the magnetic particles once again by inserting the reaction vessel into the magnetic separation module. Once again remove the supernatant by means of a disposable pipette or by extracting the magnetic block and simply decanting into a waste bottle for disposal.

5. Drying the Magnetic Particles:

For this purpose insert the reaction vessel into the heating block once again and incubate for a few minutes with the cover open at a temperature of 30° C.-70° C. For example, the incubation may be performed at a temperature of 30, 31, 32, 35, 37, 50, 42, 45, 47, 50, 55, 60, 62, 65, 68, 69, or 70° C.

6. Elution of the Nucleic Acid From the Magnetic Particles:

For this purpose use a disposable pipette or dropper bottle to add an elution buffer or to add water to the reaction vessel containing the dried magnetic particles. Shake the reaction vessel briefly and separate the magnetic particles once again by inserting the reaction vessel into the magnetic separation module. The supernatant contains the isolated nucleic acid and can be transferred into a clean reaction vessel.

In the case of some alternative embodiments of the invention, there is no need to exchange the reaction vessel between the heating and magnetic separation modules. In this case the accumulation of the magnetic particles is achieved by inserting individual magnets into the recess next to the combined heating and magnetic separation module or by energizing the electromagnet.

The isolated nucleic acid is now immediately available for the scheduled analysis procedures.

The simple handling of the hand-held unit in combination with ready-to-use extraction reagents and easy-to-operate plastic accessories therefore makes it possible to conduct isolation of nucleic acids under field conditions. Furthermore, the simplicity of the necessary sequences of the procedure allows the isolation of nucleic acids to be achieved by “non-specialists”. Above and beyond this, the isolation of nucleic acids can be achieved in principle from any sample of interest. The specific requirement is met by specific reagent kits. In this connection, the hand-held unit remains the constant component.

By virtue of the technical simplicity, the inventive hand-held unit makes a very inexpensive equipment system available for mobile isolation of nucleic acids. Such a complex hand-held unit can therefore be used in the most ideal way for diagnostic tests in developing countries. Furthermore, the universality of the hand-held unit also permits isolation of nucleic acids under real “field conditions”, for example in the context of military medical indications. In this connection, isolation of nucleic acids with the inventive hand-held unit can be achieved universally and independently of the starting sample, and the isolated nucleic acid can then be injected, for example, into portable diagnostic instruments. Portable diagnostic instruments may be packaged or carried along with the portable hand-held unit of the invention or may integrated with it. Some embodiments may contain means for detecting, recording, or communicating the detection of a target nucleic acid isolated with the hand-held unit. Some embodiments may contain means for calculating, analyzing and/or displaying a diagnostic result or risk assessment based on data obtained from the detection of one or more target nucleic acids in the isolated nucleic acid. These may include simple mechanical display materials such as colorimetric paper or plastic test strips or spots, visible, or luminescent signal, electronic display devices, or devices producing an auditory signal. Results obtained from nucleic acids isolated using the hand-held unit of the invention may be communicated electronically, via computer, radio, video or other electromagnetic transmission device. Test data or results may be communicated to computer software or hardware useful for processing test result(s) or data into information useful for diagnosis or risk assessment.

The following examples depict the possible construction of the hand-held unit for mobile isolation of nucleic acids, but are in no way to be construed as limitative.

### EXAMPLE 1

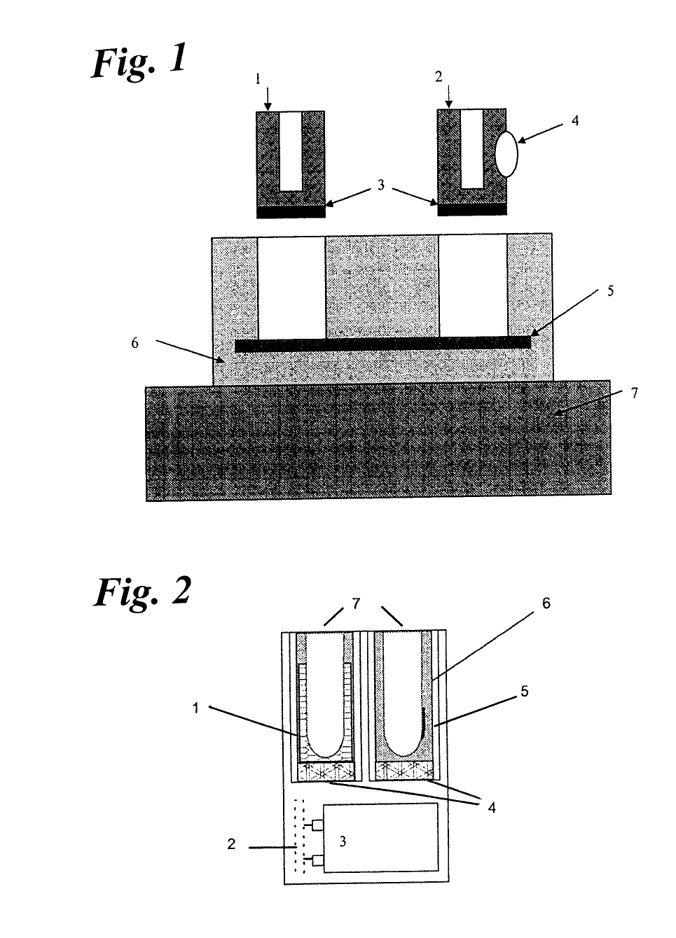

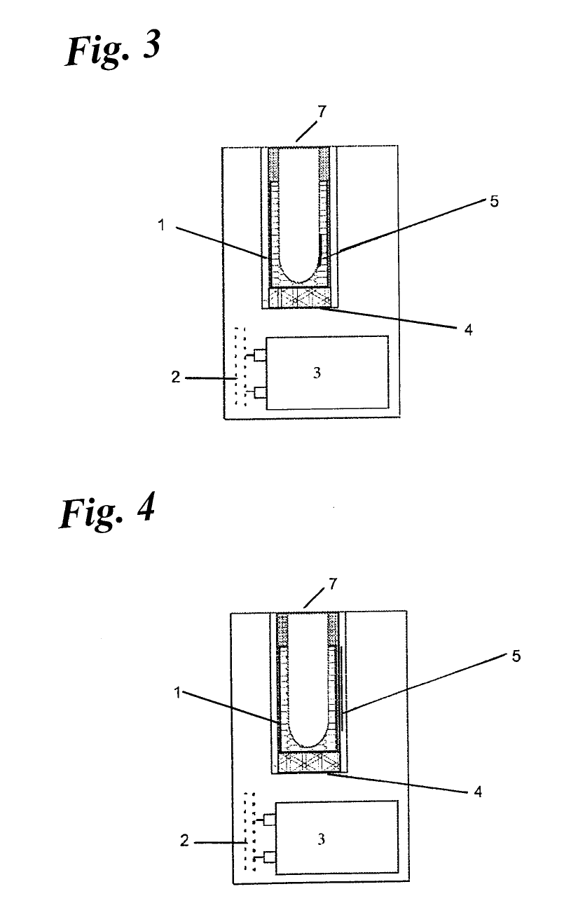

FIG. 2 shows a schematic diagram of a portable hand-held unit for isolation of nucleic acids in the case of mobile on-the-spot isolation of nucleic acids and of a test kit for performing the extraction procedure. FIGS. 3 and 4 show an alternative embodiment.

The test kit for on-the-spot isolation of nucleic acids by means of the hand-held unit contains at least one of:
1. A disposable pipette (such as a Pasteur pipette)
2. Bottle for reagent waste
3. Suspension containing magnetic or paramagnetic particles
4. Reaction vessel
5. Lysis buffer as well as optional proteolytic enzyme in liquid form or reaction vessel for lysis of the sample containing storage-stable solid formulations of lysis reagents and proteolytic enzymes
6. Binding buffer
7. Detergent buffer
8. Elution buffer.
   The test kit may contain multiples of elements 1.-8., for example, two or more disposable pipettes, two or more bottles for reagent waste, two or more reaction vessels, etc.

Preferably all extraction reagents and the needed disposable pipettes are housed in a carrying case.

Specific on-the-spot isolation of nucleic acids is performed by means of the inventive hand-held unit in the described sequence of operations.

### EXAMPLE 2

FIG. 1 shows the inventive device in a preferred embodiment. The heating block and the magnetic separation block can be removed from the sample block holder. The sample block holder contains a shaking device and if necessary heating elements and can be removed from the base device. The table below shows some differences between the embodiments shown in FIGS. 1, 2, 3 and 4,

### LIST OF REFERENCE NUMERALS

FIG. 1:
(1) Heating block
(2) Magnetic separation block
(3) heating device
(4) Magnet
(5) Shaking device, if necessary with heating element
(6) Sample block holder
(7) Device base

| FIG. 2 | FIG. 3 | FIG. 4 |
|---|---|---|
| (1) Heating mod- | (1) Separation and heating module with heating element | |
| ule with heating | | |
| element | | |
| (2) Printed circuit | | |
| board | | |
| (3) Accumulator/ | | |
| battery | | |
| (4) Vibration plate | | |
| (5) Magnetic strip | (5) Energizable electromagnet | (5) Insertable magnet |
| (6) Separation | | |
| module | | |
| (7) Recess for | | |
| reaction vessel | | |

Various modifications and variations of the described apparatus, device, systems, primers, probes, markers, other system elements, and methods of their configuration; and methods of use, including a method for processing an analyte, as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the mechanical, electronic, computational, biomedical engineering, medical, diagnostic, biological, chemical or pharmacological arts or related fields are intended to be within the scope of the following claims.

Each document, patent, patent application or patent publication cited by or referred to in this disclosure is incorporated by reference in its entirety, especially with respect to the specific subject matter surrounding the cited reference. In the event of conflict in the definition of a term, the term as described in the disclosure will control. No admission is made that any such reference constitutes background art and the right to challenge the accuracy and pertinence of the cited documents is reserved.

1. A hand-held unit for mobile isolation of at least one nucleic acid, comprising:
at least one sample block for insertion of sample vessels,
a sample block holder with bores or recesses for receiving the sample blocks,
a device base with electronic control units for the sample blocks,
a voltage supply,
a connection to the sample block holder, and,
a kit or reagent(s) for the extraction and/or isolation of nucleic acids;
wherein the sample blocks can be removed from the sample block holder, and
wherein the sample block holder can be removed from the device base.

2. The hand-held unit according to claim 1, wherein the sample blocks contain at least one heating module and/or one magnetic separation module.

3. The hand-held unit according to claim 2, which contains a magnetic separation module that is equipped with one or more magnets.

4. The hand-held unit according to claim 1, wherein a viewing window is disposed in the sample block.

5. The hand-held unit according to claim 1, wherein the sample block holder contains a shaking or vibrating device as well as one or more reversible connection points for the exchangeable blocks.

6. The hand-held unit according to claim 1, wherein the sample block holder contains an additional heating device.

7. The hand-held unit according to claim 1, wherein the sample blocks are connected to the sample block holder and the sample block holder is connected to the device base via plug connectors or a magnetic connection.

8. The hand-held unit according to claim 1, wherein the voltage supply is a battery or accumulator.

9. A hand-held unit according to claim 1, further comprising a port for an external voltage supply.

10. A hand-held unit according to claim 1, which has the following dimensions:
a height between 6 and 10 cm,
a depth between 2 and 4 cm, and
a width between 4 and 8 cm.

11. The hand-held unit according to claim 1, which has the following dimensions:
a height between 7.5 to 8.5 cm, preferably 8 cm,
depth between 2.5 to 3.5 cm, preferably 3 cm, and
width between 4.5 to 5.5 cm, preferably 5 cm.

12. The hand-held unit according claim 1, comprising
a heating module that simultaneously serves as a magnetic separation module; wherein
a) a recess for receiving a magnet is disposed next to this heating and magnetic separation module, or
b) the heating and magnetic separation module contains an energizable electromagnet.

13. The hand-held unit according to claim 1, comprising at least one of the following elements:
a) a Pasteur pipette or other disposable pipette,
b) one bottle for reagent waste,
c) a suspension containing magnetic or paramagnetic particles,
d) a reaction vessel,
e) lysis buffer as well as optional proteolytic enzyme in liquid form or reaction vessel for lysis of the sample containing storage-stable solid formulations of lysis reagents and proteolytic enzymes,
f) binding buffer,
g) detergent buffer, and
h) elution buffer.

14. The hand-held unit according to claim 13 comprising a), b), c) and d).

15. The hand-held unit according to claim 13 comprising a), b), c), d), e), f), g) and h).

16. The hand-held unit according to claim 13, comprising e) a lysis buffer and a proteolytic enzyme in liquid form.

17. The hand-held unit according to claim 13, comprising e) a lysis buffer and a reaction vessel for lysis of the sample containing storage-stable solid formulations of lysis reagents and proteolytic enzymes.

18. The hand-held unit according to claim 1, further comprising at least one means for detecting a target nucleic acid in a nucleic acid isolated by said unit, and means for recording or communicating a result of said detection.

19. A carrying container, which contains the hand-held unit according to claim 1 and, optionally, at least one portable diagnostic kit or instrument.

20. A method for mobile isolation of nucleic acids by means of the hand-held unit according to claim 1, comprising the following steps:
lysing a sample containing a nucleic acid,
binding DNA or other nucleic acid in the lysed sample to magnetic particles;
separating the magnetic particles containing the bound DNA or other nucleic acid;
washing the magnetic particles containing the bound nucleic acid;
drying the magnetic particles;
eluting the DNA or other nucleic acid from the magnetic particles; thus
recovering an isolated nucleic acid sample; and optionally
detecting a target nucleic acid sequence in said isolated nucleic acid sample.
